(19) United States
(12) Patent Application Publication
(10) Pub. No.: US 2011/0189106 A1
(43) Pub. Date: Aug. 4, 2011

(54) INTRANASAL COMPOSITIONS, DOSAGE FORMS AND METHODS OF TREATMENT

(75) Inventors: Melvyn R. Danzig, Morganville, NJ (US); Ariel Teper, Bronx, NY (US); Davis F. Gates, Summit, NJ (US); Heribert W. Staudinger, Green Brook, NJ (US)

(73) Assignee: Schering Corporation, Kenilworth, NJ (US)

(21) Appl. No.: 13/054,242

(22) PCT Filed: Jul. 13, 2009
(86) PCT No.: PCT/US2009/050361

§ 371 Date: Apr. 8, 2011

Related U.S. Application Data
(60) Provisional application No. 61/080,990, filed on Jul. 15, 2008.
(60) Provisional application No. 61/116,721, filed on Nov. 21, 2008.

(51) Int. Cl. A61K 9/12; A61K 31/56; A61P 37/08; A61P 29/00

(52) U.S. Cl. 424/45; 514/171

(57) ABSTRACT

Various embodiments of the present invention provide for compositions, dosage forms and methods useful in treating, relieving or prophylactically treating of one or more symptom of an allergic and/or inflammatory condition.

### FIELD OF THE INVENTION

The present invention relates to compositions, dosage forms and methods useful in treating, relieving or prophylactically treating of one or more symptom associated with an allergic and/or inflammatory condition.

### BACKGROUND OF THE INVENTION

Upper airway conditions such as inflammatory conditions or allergic rhinitis affect a large amount of the population. The burden of both seasonal and perennial allergic rhinitis is considerable. Allergic rhinitis can substantially decrease the quality of life and impair social and work functioning, either directly by virtue of its symptoms, or indirectly because of the inadequate relief often provided by current medications or by adverse effects of medications taken for symptom relief. Patients with allergic rhinitis often suffer from nasal congestion and, for these patients, nasal congestion can be a dominant and distressing symptom. Nasal congestion is linked to disturbances in sleep, decreased work productivity, and emotional disturbances, including discomfort and frustration. In fact, nasal congestion is one of the top reasons for a doctor's office visit.

Current treatments of nasal congestion include antihistamines, decongestants, steroids, saline, and herbal remedies. Antihistamines block the binding of the histamine mediator cells to histamine receptors of the nasal mucosa and preempt the swelling of nasal membranes, sneezing, and increased nasal secretions associated with histamine release. Antihistamines are not indicated for treating or relieving nasal congestion. Decongestants act to constrict blood vessels in the nasal mucosa and thereby decrease tissue swelling and nasal congestion. Steroids similarly reduce inflammation of swollen nasal mucosa. Treatments such as saline and herbal remedies add moisture and increase comfort but without actually relieving the congestion.

Nasal decongestants typically have a very fast onset of action and can be effective, however, are limited in use due to side effects and other problems. Oral decongestant products, such as pseudoephedrine, have side effects and the potential for conversion to illegal substances. Intranasal decongestants, such as oxymetazoline, are prescribed for twice daily dosing and typically have a very fast onset of action for relieving nasal congestion. However, use of decongestants may be limited beyond three days due to tachyphylaxis and other side effects. Prolonged use of intranasal decongestants may cause tachyphylaxis wherein the patient will need more frequent or higher doses of the decongestant to provide the same decongestion effect. Often, patients may start taking a larger than directed dose (“over-medication” or “over-dosage”) in the theory that more doses will provide more relief. While the over-medication may temporarily improve the congestion, side effects of over-medication or prolonged use include significant “rebounding” congestion after the use of the intranasal decongestant is stopped. Additionally, use of a decongestant nasal spray may lead to rhinitis medicamentosa, which is an inflammatory hypertrophy of the nasal mucosa where the tissues of the sinuses may become damaged, swollen and congested.

Intranasal corticosteroids exert a range of effects that inhibit mucosal inflammation, including (1) reducing inflammatory cell infiltration, (2) decreasing the number of basophils, eosinophils, neutrophils and mast cells in the nasal passages and their secretions, (3) reducing release of inflammatory signals from cells, (4) decreasing mucus production, (5) vasoconstriction and (6) reducing edema. Many intranasal corticosteroids are prescribed for once daily dosing and may have a longer onset of action than decongestants.

Accordingly, it would be desirable to provide an efficacious medicament for relieving or treating conditions of the upper airway passages that has a fast onset of action and can be used over a prolonged period of time with minimal side effects.

### SUMMARY OF THE INVENTION

Various embodiments of the present invention provide for an efficacious medicaments, compositions, dosage forms and methods thereof that are surprisingly efficacious in treating or relieving symptoms of allergic rhinitis and/or conditions at a once daily dose, and desirably have a fast onset of action, with minimal side effects and can be used for extended periods of time. For example, these methods may provide treatment, relief or prophylactic treatment of one or more symptom of an allergic or inflammatory condition. One or more symptom includes nasal symptoms, such as rhinorrhea, such as nasal discharge/runny nose/post-nasal drip, nasal congestion/stuffiness, sneezing as well as non-nasal symptoms, which include, nasal itching, eye watering/tearing, eye redness, eye itching, eye burning, itching of palate/ears. Various embodiments also provide for the prophylactic treatment of one or more symptom of an allergic or inflammatory condition, such as seasonal allergic rhinitis and or perennial allergic rhinitis or nasal polyposis.

Several embodiments of the present invention provide for a method of treating or relieving one or more symptom of an allergic or inflammatory condition including administering once daily to a patient in need of such treating or relieving, a therapeutically effective amount of a decongestant and a corticosteroid. For example, these methods may provide treatment or relief of symptoms such total nasal symptoms, which include rhinorrhea, such as nasal discharge/runny nose/post-nasal drip, nasal congestion/stuffiness, sneezing as well as total non-nasal symptoms, which include, nasal itching, eye watering/tearing, eye redness, eye itching, eye burning, itching of palate/ears.

Several embodiments of the present invention provide for a method of treating or relieving one or more allergic or inflammatory conditions including administering once daily to a patient in need of such treating or relieving, a therapeutically effective amount of a decongestant and a corticosteroid.

Additional embodiments of the present invention provide for methods of treating or relieving one or more symptom of an allergic or inflammatory condition including administering once daily to a patient in need of such treating or relieving, a therapeutically effective amount of a decongestant and corticosteroid in a single dosage form.

Further embodiments provide for methods of treating or relieving one or more symptom of an allergic or inflammatory condition including administering once daily to a patient in need of such treating or relieving, a therapeutically effective amount of oxymetazoline and mometasone furoate or pharmaceutically acceptable salts or polymorphs thereof.

Other embodiments provide the use of an aqueous suspension of mometasone furoate and a solution of oxymetazoline in the manufacture of a medicament for the treatment of nasal symptoms associated with allergic rhinitis, either seasonal allergic rhinitis and/or perennial allergic rhinitis.

Still other embodiments provide for a dosage form useful for treating or relieving one or more symptom of an allergic or inflammatory condition in a patient in need of treating or relieving of the conditions, the dosage form including a therapeutically effective amount of a decongestant and corticosteroid in a single dosage form. The dosage form desirably is suitable for intranasal administration.

Other embodiments provide for an intranasal pharmaceutical composition including a once daily therapeutically effective amount of oxymetazoline and mometasone furoate or pharmaceutically acceptable salts or polymorphs thereof in a single dosage form suitable for intranasal administration. The oxymetazoline or pharmaceutically acceptable salts or polymorphs thereof may be in a range of about 12.5 mcg to about 600 mcg and the mometasone furoate may be in a range from about 100 mcg to about 800 mcg. Alternatively, the oxymetazoline or pharmaceutically acceptable salts or polymorphs thereof may be in a range of about 50 mcg to about 300 mcg and the mometasone furoate or pharmaceutically acceptable salts or polymorphs thereof is in a range from about 100 mcg to about 400 mcg. The amount of oxymetazoline represents a substantially lower dose than is currently prescribed.

Further embodiments provide for a pharmaceutical product which includes a nasal spray container capable of delivering a once daily therapeutically effective amount of mometasone furoate and oxymetazoline or pharmaceutically acceptable salts or polymorphs thereof.

Other embodiments provide for a pharmaceutical composition that includes a decongestant and a corticosteroid in a once daily therapeutically effective dose useful for an administration period of at least 5 days without experiencing tachyphylaxis of the decongestant during the administration period. Other embodiments directed to methods of treating or relieving one or more symptoms of an allergic or inflammatory condition comprising administering intranasally a decongestant and a corticosteroid in a once daily therapeutically effective dose for an administration period of at least 5 days without experiencing tachyphylaxis associated with the decongestant during the administration period. Rebound is not observed after discontinuance of the administration of the decongestant and corticosteroid even after about 5 days of discontinuance of the administration of the decongestant and corticosteroid.

Further embodiments provide for an intranasal pharmaceutical composition that includes a once daily therapeutically effective amount of oxymetazoline and mometasone furoate or pharmaceutically acceptable salts or polymorphs thereof, in a single dosage form; wherein the oxymetazoline is in a range of about 50 mcg to about 300 mcg and the mometasone furoate or mometasone furoate monohydrate is in a range from about 100 mcg to about 400 mcg.

Still further embodiments provide for a method of treating or relieving one or more symptoms of an allergic or inflammatory condition in a patient in need of such treatment or relieving comprising administering intranasally a decongestant and a corticosteroid to the patient in a once daily therapeutically effective dose for an administration period of at least 5 days; wherein rebound is not observed after discontinuance of the administration of the decongestant and corticosteroid. Additionally, tachyphylaxis does not occur during the administration period and rebound is not observed after at least about 5 days after discontinuance of the administration of the decongestant and corticosteroid. The allergic or inflammatory conditions include nasal and non-nasal symptoms, such as ocular symptoms. The decongestant may be oxymetazoline or pharmaceutically acceptable salts or polymorphs thereof and the corticosteroid may be mometasone furoate or pharmaceutically acceptable salts or polymorphs thereof.

Additional embodiments provide for methods of treating or relieving one or more symptom of an allergic or inflammatory condition, such as one or more nasal or non-nasal symptom associated with seasonal allergic rhinitis, including the step of administering intranasally once daily dose to a patient in need of such treating, a therapeutically effective amount of oxymetazoline and mometasone furoate or pharmaceutically acceptable salts or polymorphs thereof. The oxymetazoline may be in a range of about 50 mcg to about 300 mcg and the mometasone furoate may be in a range from about 100 mcg to about 400 mcg. The oxymetazoline and mometasone furoate or pharmaceutically acceptable salts or polymorphs thereof may be in a single dosage form.

Additional embodiments of the present invention provide methods of treating or relieving one or more symptom associated with an allergic or inflammatory condition comprising administering intranasally once daily to a patient in need of such treating or relieving, a therapeutically effective amount of a decongestant and corticosteroid in a single dosage form. Typical symptoms include one or more symptom associated with allergic rhinitis such as nasal congestion. Still further embodiments provide methods of treating or relieving symptoms associated with one or more symptom of an allergic or inflammatory condition comprising administering intranasally once daily to a patient in need of such treating, a therapeutically effective amount of oxymetazoline and mometasone furoate or pharmaceutically acceptable salts or polymorphs thereof. Typical one or more symptoms include one or more nasal or non-nasal symptom such as those associated with seasonal allergic rhinitis or perennial allergic rhinitis. Non-nasal symptoms include one or more ocular symptoms, such as one or more symptom including eye watering, eye redness, eye itching or eye burning and combinations thereof. Nasal symptoms include one or more symptom including stuffiness, rhinorrhea, itching, and sneezing as well as nasal congestion such as when associated with allergic rhinitis.

More embodiments include a method of prophylactic treatment of one or more symptoms associated with an allergic or inflammatory condition comprising administering intranasally once daily to a patient susceptible to allergic or inflammatory condition, a therapeutically effective amount of oxymetazoline and mometasone furoate or pharmaceutically acceptable salts or polymorphs thereof, which may be in a single dosage form suitable for intranasal administration.

Other embodiments of the present invention provide methods of treating or relieving one or more symptom of an allergic or inflammatory condition including administering once daily dose to a patient in need of such treating or relieving, a therapeutically effective amount of xylometazoline or pharmaceutically acceptable salts or polymorphs thereof and a corticosteroid, such as mometasone or fluticasone or pharmaceutically acceptable salts or polymorphs thereof, such as mometasone furoate anhydrous, mometasone furoate monohydrate (MFM), fluticasone propionate or fluticasone furoate. Still other embodiments provide an intranasal pharmaceutical composition including a once daily therapeutically effective amount of xylometazoline and mometasone furoate or pharmaceutically acceptable salts or polymorphs thereof in a single dosage form suitable for intranasal administration. Further embodiments provide methods of treating or relieving one or more symptom of an allergic or inflammatory condition including administering intranasally once daily dose to a patient in need of such treating, a therapeutically effective amount of xylometazoline and mometasone furoate or pharmaceutically acceptable salts or polymorphs thereof; the xylometazoline is in a range of about 100 mcg to about 600 mcg and the mometasone furoate is in a range from about 100 mcg to about 400 mcg. Still further embodiments provide methods of treating or relieving one or more symptoms associated with an allergic or inflammatory condition including administering intranasally once daily to a patient in need of such treating or relieving, a therapeutically effective amount of xylometazoline and mometasone furoate or pharmaceutically acceptable salts or polymorphs thereof. The administration may be in a single dosage form.

### BRIEF DESCRIPTION OF DRAWINGS

FIG. 1 Change from baseline AM/PM NOW TNSS at Days 1-15.

FIG. 2 Change from baseline standardized AUC (0-4 hours) congestion at day 1.

FIG. 3 Change from baseline standardized AUC (0-4 hours) congestion at days 1 and 15.

FIG. 4 AM NOW change from baseline TNSS at days 2-15.

FIG. 5 AM NOW change from baseline congestion at days 2-15.

FIG. 6 Subject evaluation of overall condition by visit.

FIG. 7 Chart showing tachyphylaxis, if any, as shown from day 1 to day 15.

FIG. 8 Change from baseline AM/PM NOW Congestion at Days 1-15 and Days 16-22 to assess a rebound effect.

FIG. 9 Change from Baseline AM/PM PRIOR TOSS at Days 1-15

FIG. 10 Change from Baseline AM/PM PRIOR Eye Redness at Days 1-15

FIG. 11 Change from Baseline AM/PM PRIOR Eye Tearing at Days 1-15

FIG. 12 Change from Baseline AM/PM PRIOR Itching/Burning Eyes at Days 1-15

### DETAILED DESCRIPTION

Various embodiments of the present invention provide for compositions, dosage forms and methods that are surprisingly efficacious for treating one or more symptom of an allergic or inflammatory condition and that can be used for extended periods of time with minimal or a low amount of side effects.

Intranasal decongestant oxymetazoline is indicated for twice daily dosing. The present invention surprisingly found that when an intranasal decongestant is combined with an intranasal corticosteroid, the combination can be administered once daily and still be efficacious. Accordingly, several embodiments of the present invention provide for methods, compositions and dosage forms that include a decongestant and corticosteroid that are administered once daily.

Further, it has been found that when combined with an intranasal corticosteroid, an intranasal decongestant can be dosed at a lower level than currently prescribed dosing levels and still be efficacious. For instance, oxymetazoline has a typical dosing regimen of 2-3 sprays of a 0.05% nasal spray solution twice daily with a total maximum recommended dose of about 600 micrograms (mcg). The present invention surprisingly found that substantially lower daily doses of a decongestant are efficacious when combined with a corticosteroid. Additionally, the combinations were more efficacious than each of its components when given as mono therapy.

Still further, it has been surprisingly discovered that an intranasal decongestant when combined with an intranasal corticosteroid can be prescribed for a longer period of time than the current dosing recommendation for an intranasal decongestant mono therapy without experiencing tachyphylaxis, congestion rebound and/or rhinitis medicamentosa.

The present invention provides for methods, compositions and dosage forms that advantageously have a fast onset of action and a sustained effect over a day with minimal or no side effects.

It is known that patients using intranasal decongestants may experience tachyphylaxis resulting in the need for more frequent or higher doses to provide adequate decongestion. Intranasal decongestants, such as oxymetazoline, are not indicated for treatment for more than three days due in part to minimize tachyphylaxis as well as the other known side effects. Thus, enhanced and sustained efficacy over a long period of time, e.g. at least 5 days, of a decongestant when combined with a corticosteroid over the decongestant when given as mono therapy is surprising since it would not be expected that a intranasal decongestant would have a benefit at the same dosing level for such a long period of time.

Side effects were surprisingly minimal when a combination of a corticosteroid and decongestant were administered to patients. The incidence of rebound congestion due to the decongestant was not observed after several days of discontinuance of a 15 day treatment cycle. Additionally, rhinitis medicamentosa was not observed over a 15 day treatment cycle.

The decongestant and corticosteroid may be administered concomitantly or sequentially in two separate dosage forms or together in one single dosage form. The administration of the dosage forms may be in the morning or the evening.

Administration for certain types of allergic and inflammatory conditions may include a dosing regimen that includes once or twice daily dosing for a period of at least 5 consecutive days, for a period of at least 7 consecutive days, for a period of at least 10 consecutive days, for a period of at least 15 consecutive days. Alternatively, the dosing period can be once daily for about one week or about two weeks. This dosing regimen is useful for conditions such as seasonal allergic rhinitis or intermittent allergic rhinitis. Administration for other conditions will dictate a longer dosing therapy such as for six weeks to about 3 months to a dosing regimen that lasts for the full year. Such therapy is appropriate for longer term conditions such as perennial allergic rhinitis or persistent rhinitis Desirably, the efficacy of oxymetazoline does not decrease after 5 days of treatment.

Based on the judgment of the attending clinician, the effective amounts of the active pharmaceutical agents used will, of course, be dependent on the age, sex and medical history of the patient being treated and the tolerance of patient to the treatment regimen as evidenced by local toxicity (e.g., nasal irritation and/or bleeding) and by systemic side-effects (e.g. cortisol level). Cortisol is the major natural glucocorticosteroid elaborated by the adrenal cortex.

Suitable patients include those patients 12 years old and older as well as patients 2 years old to 12 years old.

Exemplary one or more symptoms of an allergic or inflammatory condition of the upper airway passages which can be treated or relieved according to various embodiments of the present invention include one or more nasal symptom associated with allergic rhinitis, such as seasonal allergic rhinitis, intermittent allergic rhinitis, persistent allergic rhinitis and/or perennial allergic rhinitis as well as congestion in moderate to severe seasonal allergic rhinitis patients. Conditions that may be treated or prevented include corticosteroid responsive diseases, nasal polyps, asthma, rhinovirus, rhinosinusitis including acute rhinosinusitis and chronic rhinosinusitis, allergic rhinitis, seasonal allergic rhinitis (SAR), perennial allergic rhinitis (PAR). Symptoms associated with these conditions include congestion, total nasal symptoms (stuffiness/congestion, rhinorrhea, nasal itching, sneezing) and non-nasal symptoms (itchy/burning eyes, tearing/watery eyes, redness of the eyes, itching of the ears/palate) and nasal blockage associated with sinusitis, fungal induced sinusitis, bacterial based sinusitis.

Examples of suitable decongestants include levmetamfetamine (also known as 1-desoxyephedrine), ephedrine, ephedrine hydrochloride, ephedrine sulfate, naphazoline, naphazoline hydrochloride, oxymetazoline or pharmaceutically acceptable salts or polymorphs thereof, oxymetazoline hydrochloride, phenylephrine, phenylpropanolamine, menazoline, phenylephrine hydrochloride, propylhexedrine, xylometazoline and xylometazoline hydrochloride or pharmaceutically acceptable salts or polymorphs thereof. Oxymetazoline is a preferred decongestant.

Useful dosing regimens for oxymetazoline for a subject may include one or two sprays once daily of a oxymetazoline solution from about 0.01% (w/v) to about 0.25%; or about 0.025% to about 0.1%; or about 0.025% to about 0.075%; or about 0.025% to about 0.05%; or 0.01%; or 0.025%; or 0.05%; or 0.075%; or 0.1%. All solution percentages are weight/volume.

Useful effective total daily amounts of oxymetazoline or pharmaceutically acceptable salts or polymorphs thereof include from about 5 to about 5000 micrograms (“mcg”)/day, from about 5 to about 2000 mcg/day, about 12.5 to about 1000 mcg/day, about 25 to about 1000 mcg/day, about 12.5 to about 800 mcg/day, about 12.5 to about 600 mcg/day, about 25 to about 500 mcg/day, 25 to about 400 micrograms, about 50 to about 500, about 50 to about 300 mcg/day, from about 50 to about 200 micrograms, from about 100 to about 300 mcg/day, about 100 mcg/day or about 200 mcg/day or about 300 mcg/day in single or divided doses. The total daily dose includes the total amount of drug delivered to both nostrils. Each nostril may receive 1 or 2 sprays.

Useful dosing regimens for xylometazoline or pharmaceutically acceptable salts or polymorphs thereof for a subject may include one or two sprays once daily of a xylometazoline solution from about 0.01% (w/v) to about 0.5%; or about 0.025% to about 0.25%; or about 0.025% to about 0.15%; or about 0.05% to about 0.1%; or 0.025%; or 0.05%; or 0.075%; or 0.1%; or 0.125%. All solution percentages are weight/volume.

Suitable corticosteroids include mometasone, dexamethasone, butoxicort, rofleponide, budesonide, deflazacort, ciclesonide, fluticasone, beclomethasone, loteprednol and triamcinolone or pharmaceutically acceptable salts or polymorphs thereof. More particularly, useful corticosteroids include mometasone furoate, mometasone furoate monohydrate, fluticasone propionate and fluticasone furoate or pharmaceutically acceptable salts or polymorphs thereof.

Mometasone furoate is a corticosteroid approved for topical dermatologic use to treat inflammatory and/or pruritic manifestations of corticosteroid-responsive dermatoses. The compound may be prepared in accordance with the procedures disclosed in U.S. Pat. Nos. 4,472,393, 4,731,447, 4,873,335, 5,837,699 and 6,127,353, all of which are hereby incorporated by reference in their entirety. Mometasone's use for the treatment of airway passages and lung diseases is disclosed in U.S. Pat. Nos. 6,677,323, 6,677,322, 6,365,581, 6,187,765, 6,068,832, 6,057,307, 5,889,015, 5,837,699 and 5,474,759, all of which are incorporated by reference in their entirety.

Useful effective total effective daily amounts of mometasone furoate or pharmaceutically acceptable salts or polymorphs thereof, such as mometasone furoate monohydrate, in suspension include from about 10 to about 5000 micrograms (“mcg”)/day, from about 10 to about 4000 mcg/day, from about 10 to about 2000 mcg/day, from about 10 to about 800 mcg/day, from about 25 to about 1000 mcg/day, from about 25 to about 400 mcg/day, from about 25 to about 200 mcg/day, from about 25 to about 100 mcg/day or from about 25 to about 50 mcg/day, from about 50 to about 800 mcg/day, from about 50 to about 200 mcg/day, from about 100 to about 200 mcg/day, about 100 or about 200 or about 300 or about 400 or about 800 mcg/day in single or divided doses.

Suitable concentrations of mometasone furoate in solution include from about 0.1 micrograms (mcg)/ml to about 500 mcg/ml; 1 mcg/ml to about 500 mcg/ml from about 5 mcg/ml to about 500 mcg/ml; 5 mcg/ml to about 250 mcg/ml; from about 5 mcg/ml to about 100 mcg/ml; from about 10 mcg/ml to about 100 mcg/ml; from about 50 mcg/ml to about 100 mcg/ml; from about 25 mcg/ml to about 75 mcg/ml; from about 50 mcg/ml to about 75 mcg/ml; from about 5 mcg/ml to about 50 mcg/ml; from about 60 mcg/ml to about 65 mcg/ml; about 5 mcg/ml; about 10 mcg/ml; about 15 mcg/ml; about 20 mcg/ml; about 25 mcg/ml; about 30 mcg/ml; about 35 mcg/ml; about 40 mcg/ml; about 45 mcg/ml; about 50 mcg/ml; about 60 mcg/ml; about 65 mcg/ml; or about 70 mcg/ml.

Useful total daily doses of mometasone in solution include, but are not limited to ranges from about 0.04 to about 100 micrograms (“mcg”)/day, about 1 to about 100 mcg/day, about 5 to about 100 mcg/day, about 5 to about 75 mcg/day, about 5 mcg to about 50 mcg/day, from about 10 mcg to about 50 mcg/day, from about 10 mcg to about 45 mcg/day, from about 10 to about 30 mcg/day, from about 40 to about 50 mcg/day, from about 15 mcg to about 25 mcg/day, from about 20 to about 25 mcg/day, about 10 mcg/day, about 15 mcg/day, 20 mcg/day, about 22.5 mcg/day, about 25 mcg/day, about 27.5 mcg/day, about 30 mcg/day about 40 mcg/day, or about 45 mcg/day.

In further example, when the corticosteroid is fluticasone or pharmaceutically acceptable salts or polymorphs thereof, it may be administered at the dose of 2 sprays of 50 μg of fluticasone propionate each in each nostril once daily. Alternatively, it may be administered at a dose of fluticasone is 1 spray of 50 μg of fluticasone propionate each in each nostril once daily. When the corticosteroid is triamcinolone or pharmaceutically acceptable salts or polymorphs thereof, it may be administered at a dose of triamcinolone is 220 μg per day as two sprays in each nostril once daily. Alternatively, it may be administered at a dose of 110 μg per day as one spray in each nostril once daily. When the corticosteroid is budesonide or pharmaceutically acceptable salts or polymorphs thereof, the administered dose of budesonide may be 64 μg per day administered as one spray per nostril of 32 μg once daily. When the corticosteroid is ciclesonide or pharmaceutically acceptable salts or polymorphs thereof, it may be administered at a dose of 200 μg per day as two sprays in each nostril once daily.

Useful amounts of mometasone include from about 0.01 to 10.0 mg, preferably 0.1 to 10.0 mg of mometasone furoate monohydrate per gram of aqueous composition. Useful amounts of oxymetazoline include between 0.025 and 10.0 mg per gram of aqueous composition.

The term “allergic rhinitis” as used herein means any allergic reaction of the nasal mucosa and includes hay fever (seasonal allergic rhinitis) and perennial rhinitis (non-seasonal allergic rhinitis) which are characterized by seasonal or perennial sneezing, rhinorrhea, nasal congestion, pruritis and eye itching, redness and tearing.

The term “non-allergic rhinitis” as used herein means eosinophilic nonallergic rhinitis which is found in patients with negative skin tests and those who have numerous eosinophils in their nasal secretions.

The term “non-malignant prolifertive and/or inflammatory disease” as used herein in reference to the pulmonary system means one or more of (1) alveolitis, such as extrinsic allergic alveolitis, and drug toxicity such as caused by, e.g. cytotoxic and/or alkylating agents; (2) vasculitis such as Wegener's granulomatosis, allergic granulomatosis, pulmonary hemangiomatosis and idiopathic pulmonary fibrosis, chronic eosinophilic pneumonia, eosinophilic granuloma and sarcoidoses.

The term “pharmaceutically acceptable salt” refers to a non-toxic salt prepared from pharmaceutically acceptable acids or bases including inorganic acids, inorganic bases, organic acids, and organic bases. Examples of suitable inorganic acids are hydrochloric, hydrobromic, hydroiodic, sulfuric, and phosphoric acid. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic acid. Examples of suitable inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine and procaine.

The phrase “therapeutically effective amount” means that amount of a medicament which when administered supplies an amount of one or more pharmaceutically active agents contained therein to provide a therapeutic benefit in the treatment or management of a disease or disease state.

Dosage form—refers to the administrable form of a medicament composition provided in a measured or unit amount, and includes at least one therapeutic agent in association with one or more other excipients comprising a delivery system, for example, a carrier, a diluent, and a coloring agent. Examples of dosage forms include, but are not limited to, gels, nasal sprays nasal drops, creams, powders, a measured amount of aerosol presented for inhalation, and a measured amount of liquid presented for imbibing.

Administration may be accomplished utilizing a device selected from a nebulizer, a metered pump-spray device, dry powder inhaler and a pressurized metered dosing inhaler. A single pressurized metered dose inhaler may be adapted for nasal inhalation routes simply by switching between an actuator that is designed for nasal delivery and an actuator designed for oral delivery.

Any suitable pump spray may be used, such as pump sprays used for NASONEX® as sold by Schering-Plough or AFRIN® as sold by Schering-Plough.

Pressurized metered-dose inhalers (“MDI”) contain propellants, for example, chlorofluorocarbon propellants, for example, CFC-11, CFC-12, hydrofluorocarbon propellants, for example, HFC-134A, HFC-227, to produce a precise quantity of an aerosol of the medicament contained with the device, which is administered by inhaling the aerosol nasally, treating the nasal mucosa and/or the sinus cavities.

The medicament formulations of the present invention may also be administered utilizing a nebulizer device. Typical commercial nebulizer devices produce dispersions of droplets in gas streams by one of two methods. Jet nebulizers use a compressed air supply to draw liquid up a tube and through an orifice by venturi action and introduce it into a flowing gas stream as droplets suspended therein, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream; these devices are less preferred for delivering suspensions.

Also available are hand-held nebulizers which atomize a liquid with a squeeze bulb air supply, but the more widely used equipment incorporates an electrically powered compressor or connects to a cylinder of compressed gas. Although the various devices which are commercially available vary considerably in their delivery efficiency for a given medicament since their respective outputs of respirable droplets are far from identical, any may be used for delivery of the medicaments of the present invention when a prescriber specifies an exact amount of medicament formulation which is to be charged to each particular device. When a nebulizer container is used to deliver for example 200 micrograms a day of an aqueous suspension of mometasone furoate, two squeezes of 50 micrograms into each nostril would normally be used to deliver the drug.

Useful aqueous compositions, such as those useful with a nasal spray, may be prepared by admixing mometasone furoate or mometasone furoate monohydrate (preferably mometasone furoate monohydrate) with water along with the decongestant and other pharmaceutically acceptable excipients. See International Application No. PCT/US91/06249 (WO 9204365); U.S. Pat. No. 6,127,353 see Examples 1-5 for preparation of mometasone furoate monohydrate and aqueous suspensions containing same. Additionally, useful compositions may be prepared in accordance with those composition described in U.S. Pat. No. 6,841,146, which is incorporated herein in its entirety.

Useful pressurized metered dose inhaler compositions may be prepared in accordance with procedures and formulations disclosed in US20040042973, U.S. Pat. No. 6,068,832, U.S. Pat. No. 6,503,482, or U.S. Pat. No. 5,474,759, which are all incorporated herein.

Aqueous compositions may contain, inter alia, water, auxiliaries and/or one or more of the excipients, such as: suspending agents, e.g., microcrystalline cellulose, sodium carboxymethylcellulose, hydroxpropyl-methyl cellulose; humectants, e.g. glycerin and propylene glycol; acids, bases or buffer substances for adjusting the pH, e.g., citric acid, sodium citrate, phosphoric acid, sodium phospate as well as mixtures of citrate and phosphate buffers; surfactants, e.g. polysorbate 80; and antimicrobial preservatives, e.g., benzalkonium chloride, phenylethyl alcohol and potassium sorbate.

Depending on the intended application, it may be desirable to incorporate up to about 10 percent by weight, more typically about 0.5 to about 5 weight percent, of an additional rheology-modifying agent, such as a polymer or other material. Useful materials include, without limitation thereto, sodium carboxymethyl cellulose, algin, carageenans, carbomers, galactomannans, hydroxypropyl methylcellulose, hydroxypropyl cellulose, polyethylene glycols, polyvinyl alcohol, polyvinylpyrrolidone, sodium carboxymethyl chitin, sodium carboxymethyl dextran, sodium carboxymethyl starch and xanthan gum. Combinations of any two or more of the foregoing are also useful.

Mixtures of microcrystalline cellulose and an alkali metal carboxyalkylcellulose are commercially available, the mixture presently preferred for use in this invention being sold by FMC Corporation, Philadelphia, Pa. U.S.A. as AVICEL® RC-591. This material contains approximately 89 weight percent microcrystalline cellulose and approximately 11 weight percent sodium carboxymethylcellulose, and is known for use as a suspending agent in preparing various pharmaceutical suspensions and emulsions. The compositions of the present invention may contain at least about 2.5 to about 10 weight percent of the mixture of the cellulose/carboxyalkylcellulose compound mixture.

A closely related mixture is available from the same source as AVICEL® RC-581, having the same bulk chemical composition as the RC-591, and this material is also useful in the invention. Microcrystalline cellulose and alkali metal carboxyalkylcellulose are commercially available separately, and can be mixed in desired proportions for use in the invention, with the amount of microcrystalline cellulose may be between about 85 and about 95 weight percent of the mixture for both separately mixed and co-processed mixtures.

When the compositions of the invention are intended for application to sensitive mucosal membranes, it will usually be desirable to adjust the pH to a relatively neutral value, using an acid or base, unless the natural pH already is suitable. In general, pH values about 4 to about 8 are preferred for tissue compatibility; the exact values chosen should also promote chemical and physical stability of the composition. In some instances, buffering agents will be included to assist with maintenance of selected pH values; typical buffers are well known in the art and include, without limitation thereto, phosphate, citrate and borate salt systems.

The compositions may contain any of a number of optional components, such as humectants, preservatives, antioxidants, chelating agents and aromatic substances. Humectants, which are hygroscopic materials such as glycerin, a polyethylene or other glycol, a polysaccharide and the like act to inhibit water loss from the composition and may add moisturizing qualities. Useful aromatic substances include camphor, menthol, eucalyptol and the like, and fragrances. Preservatives are typically incorporated to establish and maintain a freedom from pathogenic organisms; representative components include benzyl alcohol, methylparaben, propylparaben, butylparaben, chlorobutanol, phenethyl alcohol (which also is a fragrance additive), phenyl mercuric acetate and benzalkonium chloride.

Suitable medicaments that may be added to the decongestant and corticosteroid include, but are not limited to, antivirals, antihistamines, such as histamine H₁, H₂, H₃ receptor antagonists, expectorants, non-steroidal anti-inflammatory agents, anti-cholinergics, pharmaceutically acceptable zinc salts, antibiotics, leukotriene D₄ antagonists, leukotriene inhibitors, P₂Y agonists, syk kinase analogues, echinaceia, vitamin C, and vitamin E.

Examples of antibiotics useful for combining with the compositions of the present invention include macrolides, cephalosporin, and antibacterials. Specific examples of suitable antibiotics include, but are not limited to, Tetracycline, Chlortetracycline, Bacitracin, Neomycin, Polymyxin, Gramicidin, Oxytetracycline, Chloramphenicol, Florfenicol, Gentamycin, Erythromycin, Clarithromycin, Azithromycin, Tulathromycin, Cefuroxime, Ceftibuten, Ceftiofur, Cefadroxil, Amoxicillin, Peniccilins, Amoxicillin with clavulanic acid or an other suitable beta-lactamase inhibitor, Sulfonamides, Sulfacetamide, Sulfamethizole, Sulfisoxazole; Nitrofurazone, and Sodium propionate. The therapeutic amounts of compositions which may be administered are known to one of skill in the art.

Examples of Non-Steroidal Anti-Inflammatory (“NSAID's”) agents suitable for use with the present invention includes, but is not limited to, Acetylsalicylic acid, Acetaminophen, Indomethacin, Diclofenac, Piroxicam, Tenoxicam, Ibuprofen, Naproxen, Ketoprofen, Nabumetone, Ketorolac, Azapropazone, Mefenamic acid, Tolfenamic acid, Sulindac, Diflunisal, Tiaprofenic acid, Podophyllotoxin derivatives, Acemetacin, Aceclofenac, Droxicam, Oxaprozin, Floctafenine, Phenylbutazone, Proglumetacin, Flurbiprofen, Tolmetin and Fenbufen. These compositions may be administered either orally or nasally as set forth below in amounts that are known to one of skill in the art.

Pharmaceutically acceptable zinc salts contemplated for use in the present invention comprise those water soluble salts reported to have beneficial effects against the common cold. Typically such preparations comprise an aqueous or saline solution with a concentration of ionic zinc below that which causes irritation to mucus membranes. Generally the ionic zinc in such solutions is present substantially as unchelated zinc and is in the form of free ionic solution. Zinc ionic solutions for use in the present invention will typically contain substantially unchelated zinc ions in a concentration of from about 0.004 to about 0.12% (w/vol). Preferably the substantially unchelated ionic zinc compound can comprise a mineral acid salt of zinc selected from the group consisting of zinc sulfate, zinc chloride, and zinc acetate. These compositions may be administered either orally or nasally as set forth below in amounts that are known to one of skill in the art.

The invention will be further described by means of the following examples, which are not intended to limit the scope of the invention, as defined by the appended claims, in any manner.

Percentages are expressed on a weight basis, unless the context clearly indicates otherwise. The mention of any specific drug substance in this specification or in the claims is intended to encompass not only the base drug, but also pharmaceutically acceptable salts, esters, hydrates and other forms of the drug. Where a particular salt or other form of a drug is mentioned, it is contemplated that other salts or forms can be substituted.

### Examples

A study was completed to demonstrate the contribution of a nasal spray having a decongestant and a corticosteroid in the symptomatic treatment of treating allergic or inflammatory conditions of the upper airway passages, such as seasonal allergic rhinitis (SAR) and to determine the safety and the extent of tachyphylaxis and rebound congestion with the combination.

The study was a randomized, placebo-controlled, multicenter, single-blind, single-dummy, pilot study in subjects 12 years of age or older SAR. Subjects were randomized at the Baseline Visit to 15 days of treatment with either mometasone furoate nasal spray (hereinafter MFNS) 50 μg/spray, such as NASONEX® from Schering-Plough plus oxymetazoline nasal spray (0.05%) (hereinafter OXY), such as AFRIN® from Schering-Plough, concurrently administered QD (1-spray or 3-spray combination of OXY), MFNS QD, OXY BID or matching placebo nasal spray to MFNS.

This study had up to a 2-week (3 to 14 days) screening period, a 2-week (15 days) treatment period and a 1-week (7 days) post-treatment follow-up period.

All subjects who met the inclusion/exclusion criteria were randomly assigned at the Baseline Visit to 15 days of treatment with either MFNS 50 μg/spray plus OXY (0.05%) concurrently administered QD (1-spray or 3-spray combination of OXY), MFNS QD, OXY BID, or matching placebo nasal spray to MFNS. Post-baseline visits were scheduled for Days 8 and 15. A post-treatment follow-up visit was scheduled for Day 22.

Subjects evaluated, twice daily (in the AM, immediately prior to taking the morning dose, and approximately 12 hours later in the PM immediately prior to taking the evening dose), the severity of eight symptoms: rhinorrhea (nasal discharge/runny nose/post-nasal drip), nasal congestion/stuffiness, sneezing, nasal itching, eye watering/tearing, eye redness, eye itching, itching of palate/ears, on a scale of 0=none, 1=mild, 2=moderate, 3=severe; evaluation was done both as reflective over the previous 12 hours (PRIOR) and how the subject felt instantaneously at the time of evaluation (NOW). TNSS refers to the total nasal symptom score which rates the severity of rhinorrhea (nasal discharge/runny nose/post-nasal drip), nasal congestion/stuffiness, sneezing and nasal itching. TNNS refers to total non-nasal symptom score which rates the severity of eye watering/tearing, eye redness, eye itching and itching of palate/ears.

For 1 hour prior to dosing and after dosing at the baseline and Day 15 visits, subjects performed a serial evaluation of nasal congestion (NOW) for 5 hours as follows: every 15 min for 1 hr prior to dosing, every 15 min for the first hour after dosing, and then every 30 min for the next 3 hrs. The subjects remained in the office for the first 2 hrs to perform the evaluations, but performed the remaining evaluations over the next 3 hrs away from the office.

Safety evaluations included measurements of pre- and post-treatment vital signs, ECGs, laboratory parameters, and monitoring of subject-reported AEs.

The primary objective of this study was to assess the efficacy of the combination of MFNS and OXY nasal spray given concomitantly QD compared to OXY BID, MFNS QD, and placebo in subjects with SAR in relieving symptoms including nasal congestion.

A 5-arm study design was employed. The following medications were administered concomitantly as per the schedule shown below:

TABLE 1
Treatment Group of Clinical Study

| Treatment Group | AM | PM |
|---|---|---|
| Group 1 (MFNS + OXY | MFNS: 2 sprays/nostril | Matching placebo to |
| [1-spray OXY | OXY: 1 spray/nostril | MFNS: 2 |
| combination]) | | sprays/nostril |
| Group 2 (MFNS + OXY | MFNS: 2 sprays/nostril | Matching placebo to |
| [3-spray OXY | OXY: 3 sprays/nostril | MFNS: 2 |
| combination]) | | sprays/nostril |
| Group 3 (MFNS QD) | MFNS: 2 sprays/nostril | Matching placebo to |
| | Maching placebo to | MFNS: 2 |
| | MFNS: 1 spray/nostril | sprays/nostril |
| Group 4 (OXY BID) | Matching placebo to | OXY: 2 sprays/ |
| | MFNS: 2 sprays/nostril | nostril |
| | OXY: 2 sprays/nostril | |
| Group 5 (Placebo) | Matching placebo to | Matching placebo to |
| | MFNS: 2 sprays/nostril | MFNS: 2 |
| | Matching placebo to | sprays/nostril |
| | MFNS: 3 sprays/nostril | |

MFNS = mometasone furoate nasal spray;
OXY = oxymetazoline nasal spray

### Results

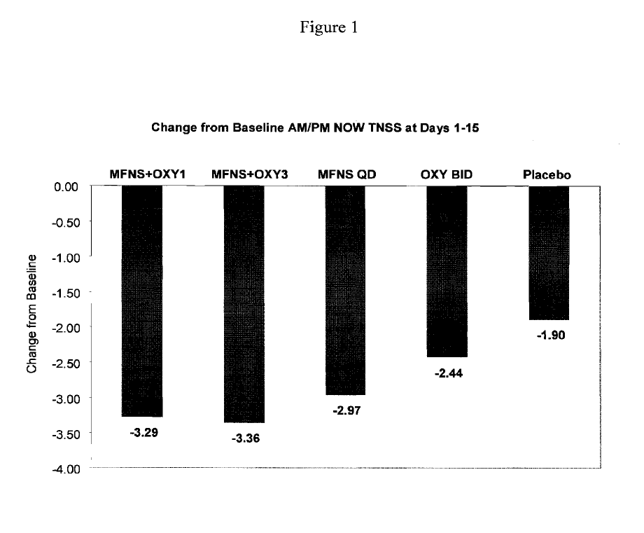

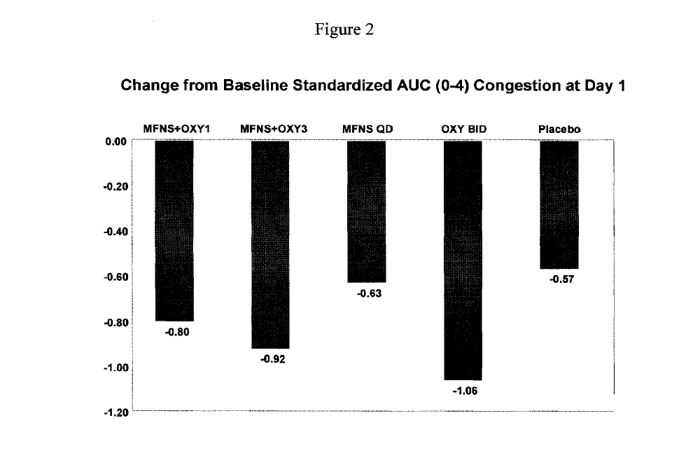

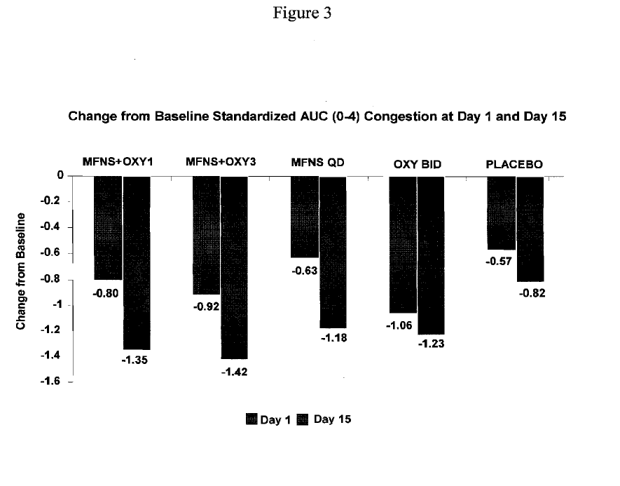

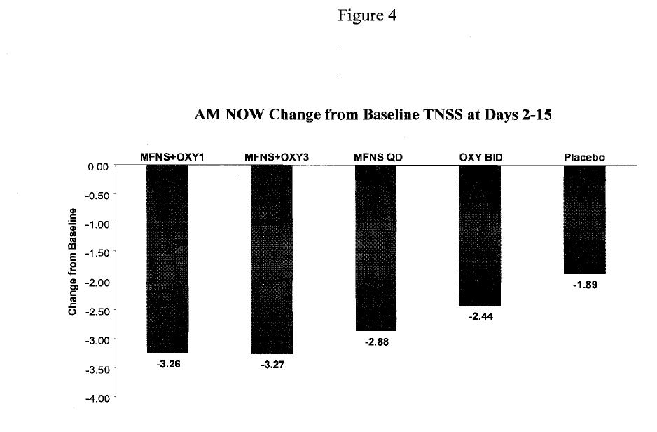

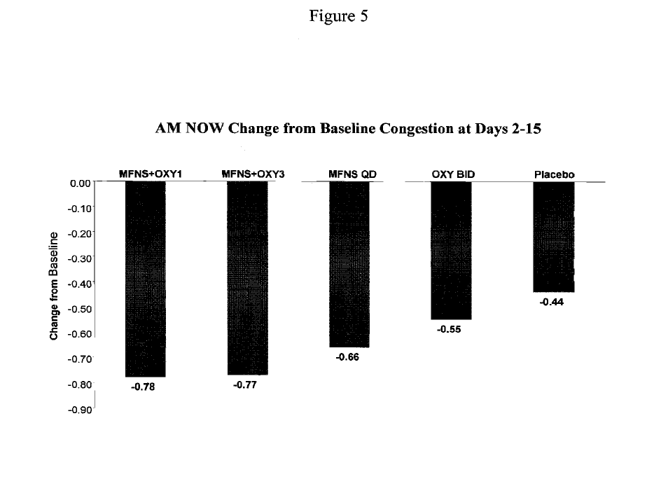

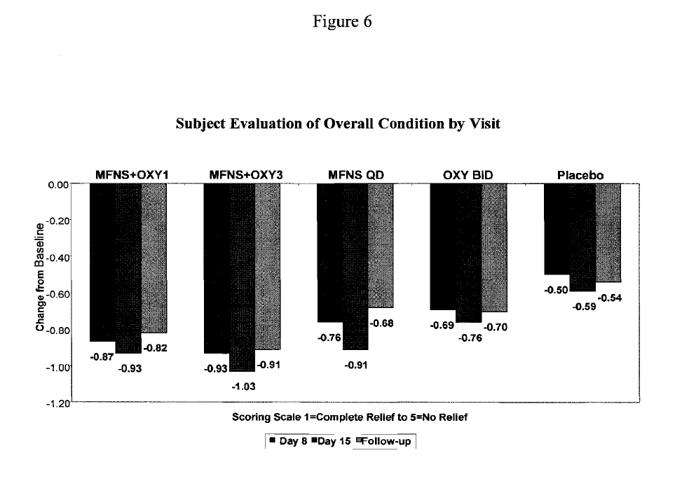

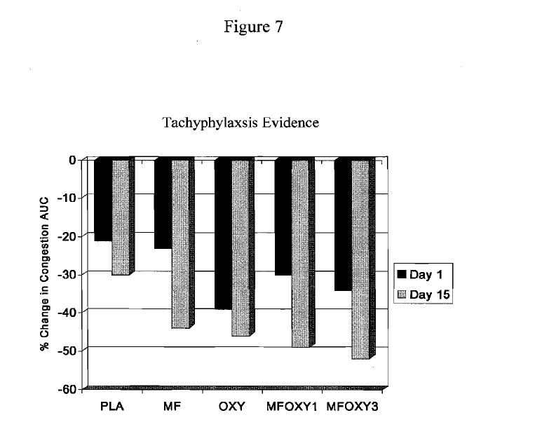

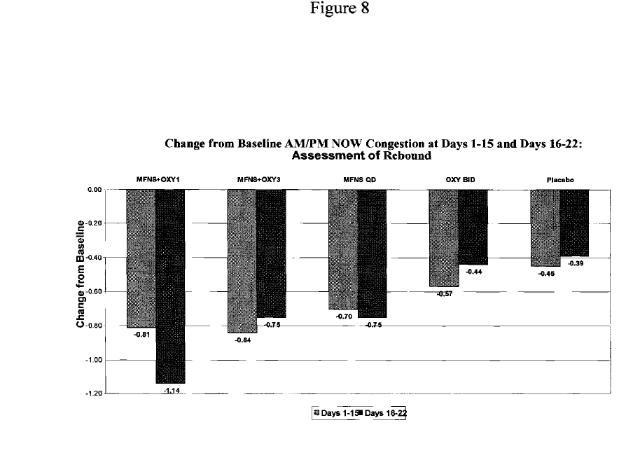

Referring to FIGS. 1-8, the combination of the corticosteroid and decongestant were superior to the decongestant alone in the AM/PM NOW TNSS over days 1-15 demonstrating the surprising enhanced effect of the combination. It was previously thought that patients using intranasal decongestants, such as oxymetazoline, experience tachyphylaxis, however, such effect was not seen with the combination of a decongestant with a corticosteroid. The change from baseline standardized AUC (0-4 hours) congestion at day 1 and day 15 was better for the combination than either component alone as given as mono therapy. This was a surprising effect exemplifying the enhanced efficacy due to the combination of components, especially when the decongestant was given at a much lower dose than it is normally prescribed and at a once daily dosing regimen. Still further, the AM NOW change from baseline for TNSS averaged over days 2 to 15 showed a surprising enhanced effect of the combination over the individual components when given as mono therapy. The AM NOW change from baseline congestion scores at days 2-15 were better for the combination than the individual components alone when given as mono therapy. As shown in FIG. 8, there appears to be no rebound effect observed during several days subsequent to stopping therapy.

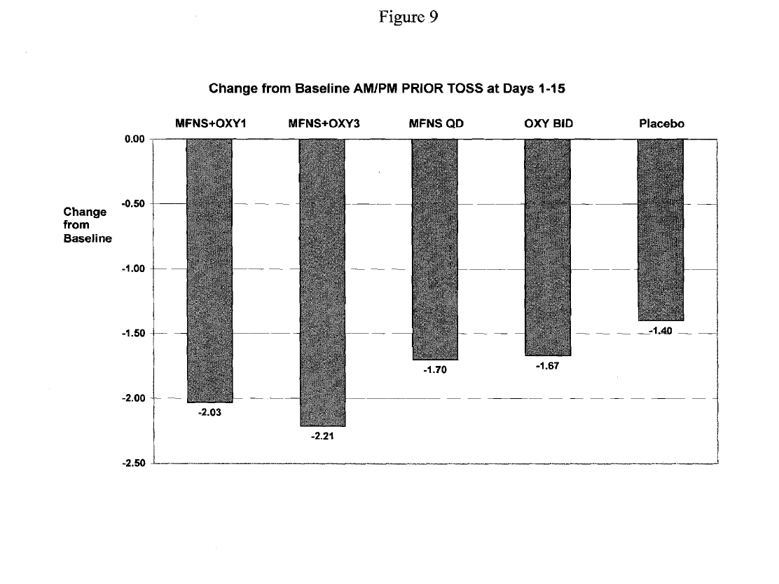

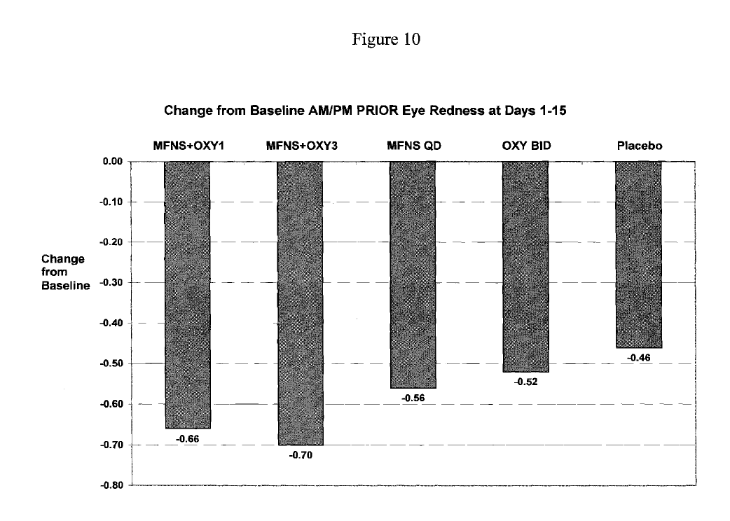

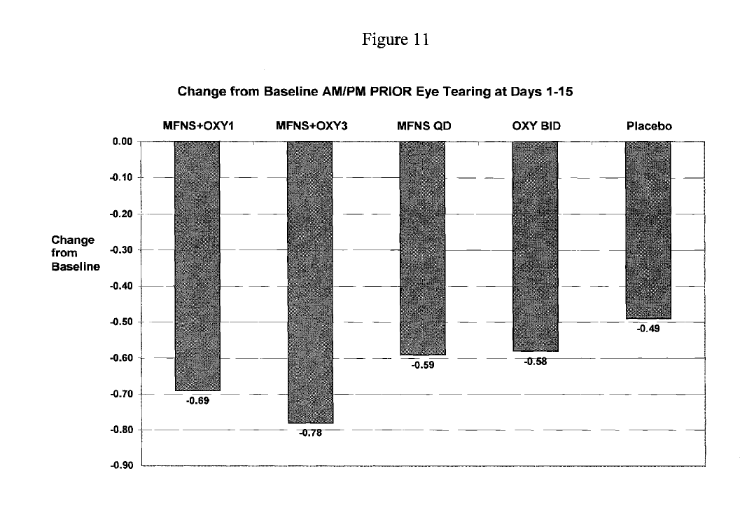

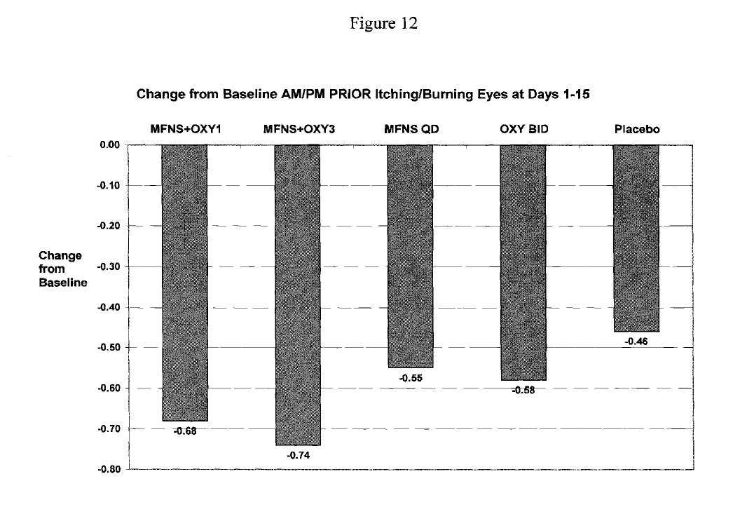

As shown in FIGS. 9-12, the compositions of the present invention have a surprisingly significant ocular effect. In particular, the combination of the corticosteroid and decongestant were superior to the decongestant alone in the AM/PM PRIOR TOSS over days 1-15 demonstrating the surprising effect of the combination. Further, there was a significant change in the AM/PM PRIOR eye redness, eye tearing and itching/burning eyes at days 1-15, as shown in FIGS. 10-12.

1. A method of treating or relieving one or more symptom of an allergic or inflammatory condition comprising intranasally administering once daily to a patient in need of such treating, a therapeutically effective amount of a decongestant and a corticosteroid.

2. The method of claim 1, wherein the therapeutically effective amount of a decongestant and corticosteroid are in a single dosage form.

3. The method of claim 1, wherein the dosage form is a nasal spray.

4. The method of claim 1, wherein the dosage form is a pressurized metered dose inhaler.

5. (canceled)

6. The method of claim 1, wherein the decongestant is selected from the group consisting of levmetamfetamine (also known as 1-desoxyephedrine), ephedrine, ephedrine hydrochloride, ephedrine sulfate, naphazoline, naphazoline hydrochloride, oxymetazoline and pharmaceutically acceptable salts thereof, oxymetazoline hydrochloride, phenylephrine, phenylpropanolamine, menazoline, phenylephrine hydrochloride, propylhexedrine, xylometazoline and xylometazoline hydrochloride or pharmaceutically acceptable salts or polymorphs thereof.

7-9. (canceled)

10. The method of claim 1, wherein the corticosteroid is selected from mometasone, dexamethasone, butoxicort, rofleponide, budesonide, deflazacort, ciclesonide, fluticasone, beclomethasone, loteprednol and triamcinolone or pharmaceutically acceptable salts or polymorphs thereof.

11-22. (canceled)

23. Method of treating or relieving one or more symptom of an allergic or inflammatory condition comprising intranasally administering a once daily dose to a patient in need of such treating or relieving, a therapeutically effective amount of oxymetazoline and mometasone furoate or pharmaceutically acceptable salts or polymorphs thereof.

24-29. (canceled)

30. The method of claim 23, wherein the administration is for a period of at least 15 consecutive days.

31-33. (canceled)

34. The method of claim 23, wherein the effective amount of mometasone furoate is in the range of about 25 to about 400 mcg/day.

35. The method of claim 23, wherein the effective amount of oxymetazoline is in the range of about 50 to about 300 mcg/day.

36. A dosage form useful for treating or relieving one or more symptom of an allergic or inflammatory condition in a patient in need of treating or relieving of the condition; the dosage form comprising a therapeutically effective amount of a decongestant and corticosteroid in a single dosage form suitable for intranasal administration.

37. The dosage form of claim 36, wherein the decongestant is selected from the group consisting of levmetamfetamine (also known as 1-desoxyephedrine), ephedrine, ephedrine hydrochloride, ephedrine sulfate, naphazoline, naphazoline hydrochloride, oxymetazoline and pharmaceutically acceptable salts thereof, oxymetazoline hydrochloride, phenylephrine, phenylpropanolamine, menazoline, phenylephrine hydrochloride, propylhexedrine, xylometazoline and xylometazoline hydrochloride or pharmaceutically acceptable salts or polymorphs thereof.

38. The dosage form of claim 36, wherein the decongestant is selected from the group consisting of phenylephrine, oxymetazoline and xylometazoline or pharmaceutically acceptable salts or polymorphs thereof.

39. The dosage form of claim 36, wherein the decongestant is oxymetazoline or pharmaceutically acceptable salts or polymorphs thereof.

40. (canceled)

41. The dosage form of claim 36, wherein the corticosteroid is selected from mometasone, dexamethasone, butoxicort, rofleponide, budesonide, deflazacort, ciclesonide, fluticasone, beclomethasone, loteprednol and triamcinolone or pharmaceutically acceptable salts or polymorphs thereof.

42. (canceled)

43. The dosage form of claim 36, wherein the corticosteroid is mometasone furoate monohydrate or mometasone furoate.

44-46. (canceled)

47. The dosage form of claim 43, wherein the effective amount of mometasone furoate is in the range from about 100 to about 400 micrograms.

48. The dosage form of claim 39, wherein the effective amount of oxymetazoline is in the range of about 12.5 to about 800 mcg/day.

49-51. (canceled)

52. The dosage form of claim 39, wherein the effective amount of oxymetazoline is in the range from about 50 to about 200 micrograms.

53-58. (canceled)

59. A method of treating or relieving one or more symptom of an allergic or inflammatory condition comprising administering intranasally once daily dose to a patient in need of such treating, a therapeutically effective amount of oxymetazoline and mometasone furoate or pharmaceutically acceptable salts or polymorphs thereof; the oxymetazoline is in a range of about 50 mcg to about 300 mcg and the mometasone furoate is in a range from about 100 mcg to about 400 mcg.

60-67. (canceled)
